(19) United States
(12) Patent Application Publication
(10) Pub. No.: US 2013/0267528 A1
(43) Pub. Date: Oct. 10, 2013

(54) TRANS-EPITHELIAL OSMOTIC COLLYRIUM FOR THE TREATMENT OF KERATOCONUS

(71) Applicant: Roberto Pinelli, Brescia (IT)

(72) Inventor: Roberto Pinelli, Brescia (IT)

(73) Assignee: AVEDRO, INC., Waltham, MA (US)

(21) Appl. No.: 13/808,515

(22) PCT Filed: Jul. 4, 2011
(86) PCT No.: PCT/IB2011/052948

§ 371 Date: Jun. 19, 2013

(30) Foreign Application Priority Data
Jul. 5, 2010 (IT) MI2010A001236

(51) Int. Cl. A61K 31/525

(52) U.S. Cl. CPC A61K 31/525
USPC 514/251

(57) ABSTRACT

Trans-epithelial osmotic collyrium for the treatment of keratoconus by corneal cross-linking with UV rays, of the type consisting of a riboflavin-based solution and an absorption enhancement adjuvant consisting of benzalkonium chloryde (BAC). The solution has a hypoosmolarity degree ranging between −70% and −30% over an isotonic solution and contains a BAC concentration accordingly ranging between 0.005% and 0.035%.

### FIELD OF THE INVENTION

The present invention concerns a collyrium for the treatment of keratoconus and, in particular, a collyrium which allows to carry out a corneal “cross-linking” treatment, without the need to provide beforehand to the removal of the corneal epithelium.

### BACKGROUND ART

Keratoconus is a progressive eye pathology, almost always a bilateral one, which determines a progressive weakening of the corneal tissue (corneal stroma, essentially consisting of water and collagen fibres) due to a remarkable reduction in the number of bonds among the collagen fibres with respect to those found in a healthy cornea. The pathology initially involves a reduction of the visual capacity and is often taken for a short-sightedness problem, while it depends instead on a change of the thicknesses—and hence of the curvature—of the cornea.

This pathology—which until a few years ago could be solved only surgically, though corneal transplant—since a few years ago can be treated with collyria containing riboflavin (vitamin B₂) or other similar compounds. As a matter of fact this substance has the property of being photosensitive and, in particular, of absorbing energy when it is struck by a suitable light radiation, then giving off such energy to surrounding water molecules which undergo hydrolysis. The ions which are released in the hydrolysis process build free radicals which bind with the collagen fibre and hence contribute to strengthen and harden the corneal stroma. A light source used in the cross-linking treatment through riboflavin is for example a source of UVA rays with a wavelength of 370 nm and a power/rating of 3 mW/cm².

For the cross-linking treatment with riboflavin to be effective it is hence essential that this substance penetrates into the corneal stroma. However, unfortunately the size of the riboflavin molecule is so big that the intact eye epithelium represents an impenetrable barrier for riboflavin, as was verified and confirmed in various studies: E. Spoerl, M. Huhle and T. Seiler: “Induction of cross-links in corneal tissue” Exp. Eye Research 1998; 66: 97-103—S. Hayes D. P. O'Brart, L. S. Lamdin, J. Doutch, K. Samaras, J. Marshall and K. M. Meek: “Effect of complete epithelial debridement before riboflavin-ultraviolet-A corneal collagen cross-linking therapy” J. Cataract Refractive Surgery 2008; 34; 657-661—S. Baiocchi, C. Mazzotta, D. Cerretani, T. Caporossi and A. Caporossi: “Corneal cross-linking: Riboflavin concentration in cornea stroma exposed with and without epithelium” J. Cataract Refractive Surgery 2009; 35: 893-899—G. Wollensak and E. Iomdina: “Biomechanical and histological changes after corneal cross-linking with and without epithelial debridement” J. Cataract Refractive Surgery 2009; 35: 540-546.

In the conventional approach therefore, before the treatment with riboflavin a full surgical removal of the eye epithelium is carried out, so as to remove the barrier effect to the riboflavin treatment. However, the epithelium removal determines post-surgery pain in the patient, while the epithelium-deprived cornea is an open wound with a high risk of infection. Although these unfavourable events may be controlled by pain-killers and antibiotics, various possible complications (various types of keratitis) have been reported in the literature. In any case, the re-epitelisation process of the treated part is rather long and often implies episodes of corneal oedema and resulting opacisation of the cornea, so that this keratoconus treatment may be excessively invasive for the patient.

For these reasons there is currently a strong interest in the field towards developing alternative keratoconus treatments which allow to treat the corneal stroma with riboflavin without requiring the prior removal of the corneal epithelium.

A first research trend has faced the problem from the point of view of a change of the surgical technique, for example through the forming of stromal pockets according to a grid pattern, through which riboflavin is caused to penetrate into the corneal tissue (R. R. Krueger, J. C. Ramos-Esteban, J. Kanellopolous, et al.: “Staged intrastromal delivery of riboflavin with UVA cross-linking in advanced bullous keratopathy: Laboratory investigation and first clinical case” J. Cataract Refractive Surgery 2008; 24: S730-S736; J. Kanellopolous: “Collagen cross-linking in early cheratoconous with riboflavin in a ferntosecond laser-created pocket: initial clinical result” J. Cataract Refractive Surgery 2009; 25: 1034-1037), or through the application of riboflavin through the front chamber of the eye (K. M. Bottos, P. Schor, J. L. Dreyfus, H. B. Nader, W. Chamon: “Corneal cross-linking with intact epithelium: immunohistological findings” submitted, waiting for publication).

A second trend has addressed instead the search of collyrium compositions capable of improving the permeability of the corneal epithelium by riboflavin, removing or reducing the barrier effect that such epithelium exhibits in standard conditions. The present invention belongs to such second trend.

WO2010/023705 by SOOFT ITALIA proposes to use a collyrium in which riboflavin is associated with a mixture of additives which improve the penetration capability into the corneal epithelium and/or photoactivity thereof, such as bioactivators and photoactivators; in the Examples hypotonic formulations with various activators are disclosed, however, without attributing any particular effect to the hypotonicity of the solution. As a matter of fact in the document it is stated that such hypotonic solutions can be indiscriminately replaced by isotonic or hypertonic solutions.

R. Pinelli, inventor of the present application, in the unpublished application EP-09006166 has described a collyrium for the treatment of keratoconus in which with riboflavin a small amount of surface-active agent is associated, preferably benzalkonium chloryde (BAC) at 0.02%, which aids penetration thereof through the corneal epithelium. In this document too the use of a hypotonic solution is proposed, however, without carrying out any in depth study or proposing any conclusion on the relevance of this condition.

G. Wollensak and E. Iomdina (2009—see above) in the above-cited publication offer a first systematic study, carried out on rabbit eyes, of the effects of the combination of riboflavin with BAC at 0.005%, detecting a slight corneal, bio-mechanical effect.

L. L. Vicente and B. S. Boxer Wachler (“Three-year study results of trans-epithelial corneal collagen cross-linking for cheratoconus” submitted, waiting for publication), proposes to use tetracaine, an anaesthetic for topic use used in ophthalmology, as a compound suitable to reduce the barrier effect of corneal epidermis, to aid riboflavin absorption.

A. Kisser, E. Spoerl, R. Jung, K. Spekl, L. E. Pillunat, F. Raiskup (“Pharmacological modification of the epithelial permeability by benzalkonium chloride in USA/riboflavin corneal collagen cross-linking” Current Eye Research; 2010—being printed) disclose a series of tests on rabbit eyes with various concentrations of BAC and conclude confirming good results in terms of riboflavin absorption through the corneal epithelium in the presence of BAC at 0.02% in a hypotonic solution, hence similar to the one disclosed by Pinelli (see above).

### PROBLEM AND SOLUTION

Following the studies carried out after the filing of the above-cited patent application, the inventor has supposed that the use of a hypo-osmolar solution, up until that moment employed in collyria for the sole purpose of determining an osmotic pressure directed towards the inside of the eye, was instead also a determining factor for obtaining increased permeability of the epithelial barrier, due to an unforeseeable widening effect of the mesh-size of the network formed by said barrier, sufficient to allow the passing of large riboflavin molecules with no need for surgical removal intervention of at least part of the epithelium. The tests subsequently carried out in this respect have then allowed to confirm this insight and to define a hypoosmolarity range in which the permeability effect of the corneal epithelium occurs in a substantial manner, thus coming to the present invention.

The object of the invention is hence that of providing a riboflavin-based collyrium composition which offers improved permeability of the active principle through the corneal epithelium and hence allows an effective treatment of keratoconus without surgical intervention for epithelium removal.

This object is achieved through a collyrium having the features defined in the claim 1 herewith enclosed. Further features of the collyrium of the invention are defined in the dependent claims.

### DETAILED DESCRIPTION OF A PREFERRED EMBODIMENT

The invention will in any case be better described now in the following, reporting as a non-limiting example, the results of some of the tests carried out.

The tests were directed at verifying the effects on the permeability of the corneal epithelium of the use of solutions with a different hypoosmolarity degree and furthermore the change of such effects following the addition of a known adjuvating agent consisting of benzalkonium chloryde.

### Preparation of Collyrium Solutions

Various collyrium solutions have then been prepared for subsequent testing, all containing the same riboflavin concentration, however, with a different degree of hypoosmolarity and different BAC concentrations. The concentrations of the different solutions are reported in Table 1 here below, where the reference example is also stated in which each solution has been used. All the concentrations are expressed as percentages in weight; the degree of hypoosmolarity of the different solutions is stated as a percentage drop of the NaCl contents over that of an isotonic solution.

| Example | Riboflavin | BAC | NaCl | Hypoosmolarity |
|---|---|---|---|---|
| Standard | 0.1% | — | 0.70% | −22% |
| Inv. 1 | 0.1% | 0.02% | 0.44% | −51% |
| Inv. 2 | 0.1% | 0.01% | 0.44% | −51% |
| Inv. 3 | 0.1% | — | 0.44% | −51% |
| Cfr. 1 | 0.1% | 0.02% | 0.90% | isotonic |
| Cfr. 2 | 0.1% | — | 0.70% | −22% |

The solution of the Standard Example is the one which today is habitually used in the corneal cross-linking treatment through the removal of the corneal epithelium, and it is a moderately hypo-osmolar solution devoid of adjuvants.

The solutions of Inv. 1, Inv. 2 and Inv. 3 Examples are all solutions according to the invention with hypoosmolarity equal to about −50% and with different BAC concentrations.

The solution of Cfr. 1 Example is an isotonic comparison solution containing 0.02% of BAC, while the solution of Cfr. 2 Example is a solution identical to the one used in the Standard Example.

### Examples of Application of the Collyrium Solutions

The above-specified collyrium solutions have been tested on groups of 6 rabbit eyes taken from animals killed for other tests. For this basic study it has hence not been necessary to sacrifice further animals.

The test collyrium solutions have been applied to the eyes after the application of a containment ring so as to protect surrounding tissues. Only in the first group (Standard Example) the corneal epithelium has been removed from the enucleated eyes, according to the standard technique currently most in use, while in the other five groups (Invention Examples and Comparison Examples) the cornea has been left intact.

The application time of the collyrium solution, in all groups, was of 30 minutes. At the end of this period, in each sample the corneo-scleral button was removed from treated eyes by drilling, placed on a slide and the absorption intensity of UV radiation under radiation of a UV source having known features was measured. Subsequently the corneal epithelium was carefully removed and a second measurement of the absorption intensity of the UV radiation was carried out, so as to have separate data of absorption in the epithelium and in the corneal stroma.

At the same time the thicknesses of the cornea were measured with a pachymeter before the application of the solution, after the 30-minute application of the riboflavin-based solution and after epithelium removal. The values of the absorption coefficient were calculated in a known manner starting from the above reported data, according to what has been indicated by Wollensak (see above).

### Test Results

The results of the tests described above are reported—for each of the Examples—in the below reported Table 2.

TABLE 2
Results of UV absorption tests

| | | | | epithelium | stroma |
|---|---|---|---|---|---|
| | | | | absorption | absorption |
| Example | Riboflavin | BAC | NaCl | coeff. cm⁻¹ | coeff. cm⁻¹ |
| Standard | 0.1% | — | 0.70% | | 41.8 |
| Inv. 1 | 0.1% | 0.02% | 0.44% | 28.3 | 17.8 |
| Inv. 2 | 0.1% | 0.01% | 0.44% | 20.0 | 15.0 |
| Inv. 3 | 0.1% | — | 0.44% | 12.7 | 11.5 |
| Cfr. 1 | 0.1% | 0.02% | 0.90% | 8.3 | 9.1 |
| Cfr. 2 | 0.1% | — | 0.70% | 5.6 | 3.6 |

From the above-reported data various conclusions can be drawn which confirm the inventor's initial insight.

By comparing the results of Invention Examples 1 and 3 with those of Comparison Examples 1 and 2 it is firstly possible to observe that—the riboflavin and BAC contents being equal—there is a substantial doubling of the absorption coefficient in the corneal stroma when one switches from an isotonic or moderately hypotonic solution to the solutions of the invention which have a hypoosmolarity equal to about −50%.

By comparing the results of Inv. 3 and Cfr. 1 Examples it is furthermore possible to observe that in the former case better results are obtained in terms of absorption, despite the absence of BAC.

Further tests, not reported in detail here, have furthermore allowed to observe that the positive effect of the osmolarity reduction on riboflavin permeability of the corneal epithelium begins to be significally revealed for hypoosmolarity values equal to about −30% (about 0.63% NaCl) and it increases, upon hypoosmolarity decrease, up to a plateau condition which is found around a hypoosmolarity value equal to about −70% (about 0.27% NaCl).

Therefore, due to this important discovery, it now becomes possible to formulate the collyrium riboflavin contents—as a function of the patient's personal conditions and in the light of the toxicity of this product—despite maintaining the same treatment effectiveness, provided one takes care to associate lower riboflavin doses with solutions of a higher hypoosmolarity degree, so that the greater permeability increase effect through the corneal epithelium, induced by these solutions, can compensate the lower concentration of the active principle. Within the above-defined hypoosmolarity range between −70% and −30% it can hence be assumed to arrange collyrium solutions having riboflavin concentrations varying according to a linear proportionality in the range between 0.005% and 0.035%, all said solutions exhibiting—at least in first approximation—a constant absorption coefficient and thus offering the physician a wide range of choices depending on the clinical conditions of the individual patient and of the progression of the disease.

However, it is understood that the invention must in no way be considered limited to the particular illustration of the same given above, which represent only a partial series of tests thereof, but that a number of variants are possible, all within the reach of a person skilled in the field, without departing from the scope of the invention, as defined by the following claims.

1. A trans-epithelial osmotic collyrium for treating keratoconus by corneal cross-linking with UV rays, comprising: a riboflavin-based solution with absorption enhancement adjuvants, wherein said solution is a hypo-osmolar solution with a hypoosmolarity degree ranging between −70% and −30% over an isotonic solution.

2. The trans-epithelial osmotic collyrium as claimed in claim 1, wherein said absorption enhancement adjuvants comprise benzalkonium chloride (BAC), in an amount ranging between 0.005% and 0.035%.

3. The trans-epithelial osmotic collyrium as claimed in claim 2, wherein said riboflavin-based solution contains a concentration of BAC that varies inversely proportionally with the degree of hypoosmolarity within said ranges.

4. (canceled)

5. A method for treating keratoconus, comprising:
applying, to a cornea of an eye without removal of the corneal epithelium, a riboflavin-based solution with absorption enhancement adjuvants, wherein said solution is a hypo-osmolar solution with a hypoosmolarity degree ranging between −70% and −30% over an isotonic solution; and
applying, to said cornea of said eye, UV rays to generate corneal cross-linking with said riboflavin-based solution with said absorption enhancement adjuvants.

6. The method as claimed in claim 5, wherein said absorption enhancement adjuvants comprise benzalkonium chloride (BAC), in an amount ranging between 0.005% and 0.035%.

7. The method as claimed in claim 6, wherein the riboflavin-based solution contains a concentration of BAC that varies inversely proportionally with the degree of hypoosmolarity within said ranges.
